(19) United States
(12) Patent Application Publication
(10) Pub. No.: US 2006/0018857 A1
(43) Pub. Date: Jan. 26, 2006

(54) Conditioning surface-active preparation

(76) Inventors: Ansgar Behler, Bottrop (DE); Hermann Hensen, Haan (DE); Werner Seipel, Hilden (DE); Iris Huetter, Kempen-Toenisforst (DE); Sybille Cornelsen, Ratingen (DE); Karl Heinz Schmid, Mettmann (DE); Josef Koester, Harleysville, PA (US); Markus Doerr, Glenmoore, PA (US)

Correspondence Address: COGNIS CORPORATION;PATENT DEPARTMENT, 300 BROOKSIDE AVENUE, AMBLER, PA, 19002, US

(21) Appl. No.: 11/187,132

(22) Filed: Jul. 22, 2005

(30) Foreign Application Priority Data
Jul. 22, 2004 (DE) 10 2004 035 633.5

(51) Int. Cl. A61K 8/60; A61K 8/365

(52) U.S. Cl. 424/70.13; 424/70.22

(57) ABSTRACT

The invention relates to a cosmetic preparation containing (a) 0.1 to 30% by weight of at least one anionic, nonionic and/or amphoteric surfactant, (b) 0.05 to 20% by weight of an α-hydroxycarboxylic acid ester of ethoxylated alcohols corresponding to formula (I):
R¹O(CH₂CH₂O)ₙH (I)
in which R¹ is a linear or branched alkyl or alkenyl group containing 6 to 22 carbon atoms and n is a number of 1 to 50, (c) 0.005 to 1.5% by weight of at least one alkaline earth metal ion and (d) 0.1 to 3% by weight of a silicone compound, and to the use of α-hydroxycarboxylic acid esters of ethoxylated alcohols as conditioning components and to reduce or prevent the absorption of alkyl ether sulfates onto the skin.

### CROSS-REFERENCE TO RELATED APPLICATIONS

This application claims priority under 35 USC § 119 from German Patent Application No. 10 2004 035 633.5, filed on Jul. 22, 2004.

### BACKGROUND OF THE INVENTION

1. Technical Field

This invention relates generally to conditioning surface-active preparations and, more particularly, to the use of long-chain alkoxylated hydroxycarboxylic acid esters as conditioning agents.

2. Background Art

Skin and hair conditioners are generally understood to be preparations which improve the properties of the skin and hair from the user's perspective. This includes improvements in the feel or luster of the hair and in the softness of the skin and hair. The smoothing of the skin and hair is particularly desirable. Cationic polymers which develop this effect on the skin and hair are generally used in surface-active formulations. For example, U.S. Pat. No. 6,264,931 describes 2-in-1 conditioners containing anionic surfactants in combination with cationic polymers. These cationic conditioners often have the disadvantage of poor biodegradability. In addition, powder-form polymers are often very difficult to incorporate homogeneously in cosmetic preparations.

However, surface-active preparations are also supposed to subject the skin and hair to minimal stress. Thus, EP 0371339 B1 describes surface-active water-based mixtures containing fatty alcohol ether sulfates and fatty alcohol ether citrates in combination with alkaline earth metal ions which subject the skin and hair to comparatively little stress.

Accordingly, the problem addressed by the present invention was to provide conditioners for the skin and hair which would have excellent properties coupled with extremely good skin and hair care activity. In addition, there would be no need for cationic conditioners to be present.

### BRIEF SUMMARY OF THE INVENTION

The present invention relates to cosmetic preparations containing
(a) 0.1 to 30% by weight of at least one anionic, nonionic and/or amphoteric surfactant,
(b) 0.05 to 20% by weight of an α-hydroxycarboxylic acid ester of ethoxylated alcohols corresponding to formula (I):
   R¹O(CH₂CH₂O)ₙH (I)
   in which R¹ is a linear or branched alkyl or alkenyl group containing 6 to 22 carbon atoms and n is a number of 1 to 50,
(c) 0.005 to 1.5% by weight of at least one alkaline earth metal ion and
(d) 0.1 to 3% by weight of a silicone compound.

### BRIEF DESCRIPTION OF THE SEVERAL VIEWS OF THE DRAWINGS

FIG. 1: Sensory Assessments—shows that skin treated with the co-surfactant of the invention, Laureth-7 Citrate, is felt to be softer than skin treated with a standard co-surfactant, Cocamidopropylbetaine.

FIG. 2: Half head test shampoo versus standard with no added polymer—shows that the preparation containing the α-hydroxycarboxylic acid ester co-surfactant of the invention (Laureth-7-citrate) has clearly superior conditioning properties to the preparation containing only a standard co-surfactant (Cocamidopropylbetaine) and no added polymer.

FIG. 3: Half head test shampoo versus standard with added polymer—shows that the preparation containing the α-hydroxycarboxylic acid ester co-surfactant of the invention (Laureth-7-citrate) leads to improved combability of dry hair and to a more pleasant feel of the hair over the standard co-surfactant (Cocamidopropylbetaine) and a polymer.

FIG. 4: Adsorption of sodium lauryl ether sulfate onto skin—shows that, where α-hydroxycarboxylic acid esters of ethoxylated alcohols of formula (I) (Laureth-7 Citrate) according to the invention are present, the adsorption of sodium lauryl ether sulfate is prevented considerably more effectively than with a standard co-surfactants (cocoglutamate).

### DETAILED DESCRIPTION OF THE INVENTION

Surfactants

Component (a) of the cosmetic preparations according to the invention is selected from anionic, nonionic and/or amphoteric/zwitterionic surfactants. Typical examples of anionic surfactants are soaps, alkyl benzenesulfonates, alkanesulfonates, olefin sulfonates, alkylether sulfonates, glycerol ether sulfonates, α-methyl ester sulfonates, sulfofatty acids, alkyl sulfates, fatty alcohol ether sulfates, glycerol ether sulfates, fatty acid ether sulfates, hydroxy mixed ether sulfates, monoglyceride (ether) sulfates, fatty acid amide (ether) sulfates, mono- and dialkyl sulfosuccinates, mono- and dialkyl sulfosuccinamates, sulfotriglycerides, amide soaps, ether carboxylic acids and salts thereof, fatty acid isethionates, fatty acid sarcosinates, fatty acid taurides, N-acylamino acids such as, for example, acyl lactylates, acyl tartrates, acyl glutamates and acyl aspartates, alkyl oligoglucoside sulfates, alkyl glucose carboxylates, protein fatty acid condensates (particularly wheat-based vegetable products) and alkyl (ether) phosphates. If the anionic surfactants contain polyglycol ether chains, they may have a conventional homolog distribution although they preferably have a narrow-range homolog distribution. Typical examples of nonionic surfactants are fatty alcohol polyglycol ethers, alkylphenol polyglycol ethers, fatty acid polyglycol esters, fatty acid amide polyglycol ethers, fatty amine polyglycol ethers, alkoxylated triglycerides, mixed ethers and mixed formals, optionally partly oxidized alk(en)yl oligoglycosides or glucuronic acid derivatives, fatty acid-N-alkyl glucamides, protein hydrolyzates (particularly wheat-based vegetable products), polyol fatty acid esters, sugar esters, sorbitan esters, polysorbates and amine oxides. If the nonionic surfactants contain polyglycol ether chains, they may have a conventional homolog distribution, although they preferably have a narrow-range homolog distribution. Typical examples of cationic surfactants are quaternary ammonium compounds, for example dimethyl distearyl ammonium chloride, and esterquats, more particularly quaternized fatty acid trialkanolamine ester salts. Typical examples of amphoteric or zwitterionic surfactants are alkylbetaines, alkylamidobetaines, aminopropionates, aminoglycinates, imidazolinium betaines and sulfobetaines.

In one preferred embodiment, alkyl ether sulfates, alkyl amidobetaines, acylated amino acids, alk(en)yl oligoglycosides, alkyl glucose carboxylates or alkyl amphoacetates are used as component (a).

The surfactants are used in the preparations according to the invention in quantities of 0.1 to 20% by weight and preferably in quantity of 9 to 20% by weight.

α-Hydroxycarboxylic Acid Esters

α-Hydroxycarboxylic acids are organic acids which, besides at least one COOH group, contain at least one OH group in the molecule. With one OH group, they may be present as monohydroxycarboxylic acids, with two OH groups as dihydroxycarboxylic acids or with more than two OH groups as polyhydroxycarboxylic acids. Hydroxycarboxylic acids are divided into alpha-, beta- and gamma-hydroxycarboxylic acids according to the position of the OH group to the COOH group. α-Hydroxycarboxylic acids preferred for the purposes of the invention are tartaric acid, mandelic acid, lactic acid, malic acid, citric acid and salts and self-condensation products thereof. Citric acid is particularly preferred for the purposes of the invention.

The α-hydroxycarboxylic acid esters are derived from ethoxylated C₆-22 alcohols corresponding to general formula (i):
R¹O(CH₂CH₂O)ₙH (I)
in which R¹ is a linear or branched alkyl or alkenyl group containing 6 to 22 carbon atoms and n is a number of 1 to 50. In formula (I), the degree of ethoxylation n is a number of 1 to 20, preferably 1 to 10 and more particularly 3 to 8. Hydroxycarboxylic acid esters derived from ethoxylated alcohols of formula (I), in which R¹ is a linear alkyl group, are particularly suitable.

Typical examples are adducts of on average 1 to 20, preferably 1 to 10 and more particularly 3 to 8 mol ethylene oxide with caproic alcohol, caprylic alcohol, 2-ethylhexyl alcohol, capric alcohol, lauryl alcohol, isotridecyl alcohol, myristyl alcohol, cetyl alcohol, palmitolelyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol and brassidyl alcohol and the technical mixtures thereof obtained, for example, in the high-pressure hydrogenation of technical methyl esters based on fats and oils or aldehydes from Roelen's oxo synthesis and as monomer fraction in the dimerization of unsaturated fatty alcohols. Adducts of 1 to 10 and more particularly 3 to 8 mol ethylene oxide with technical C₁₂-18 fatty alcohols, such as for example coconut oil, palm oil, palm kernel oil or tallow fatty alcohol.

In one embodiment of the present invention, R¹O in formula (I) is derived from a fatty alcohol mixture containing 65 to 75% by weight C₁₂, 20 to 30% by weight C₁₄, O to 5% by weight C₁₆ and 0 to 5% by weight C₁-8 alcohols. This alcohol mixture is commercially available, for example, as Lorol® Spezial from Cognis Deutschland GmbH & Co. KG. Hydroxycarboxylic acid esters based on this fatty alcohol mixture preferably have a degree of ethoxylation “n” of, on average, 4.

In another embodiment of the present invention, R¹O in formula (I) is derived from a fatty alcohol mixture containing 45 to 60% by weight C₁₂, 15 to 30% by weight C₁₄, 5 to 15% by weight C₁₆ and 8 to 20% by weight C₁₈ alcohols. This alcohol mixture is also commercially available, for example, as Lorol® from Cognis Deutschland GmbH & Co. KG. Hydroxycarboxylic acid esters based on this selected fatty alcohol mixture have preferably been ethoxylated with, on average, 7 mol ethylene oxide (n=7).

According to the invention, the α-hydroxycarboxylic acids may be completely or, more particularly, partly esterified. In the case of partial esterification, the compounds still contain at least one free carboxyl group. Accordingly, they may be esters or neutralization products thereof. The partial esters are preferably present in the form of the alkali metal, alkaline earth metal, ammonium, alkylammonium, alkanolammonium and/or glucammonium salts.

In a particularly preferred embodiment of the invention, component (b) is formed by esters of citric acid with ethoxylated alcohols corresponding to formula (I):
R¹O(CH₂CH₂O)ₙH (I)
in which R¹ is a linear or branched alkyl or alkenyl group containing 6 to 22 carbon atoms and n is a number of 1 to 50.

The most particularly preferred citric acid esters are preferably mixtures of isomeric compounds corresponding to general formula (II): 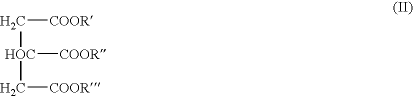
in which R′, R″, R′″ stand for X and/or an ethoxylated alkyl group R¹ with the meaning defined for formula (I), the distribution of the substituents R′, R″ and R′″ having to be such that the ratio by weight of monoester to diester is in the range from 3:1 to 10:1. In a preferred embodiment, the ratio by weight of monoester to diester is in the range from 5:1 to 8:1.

Accordingly, the preferred citric acid ester mixtures according to the invention compulsorily contain mono- and diesters, preferably in quantities of 50 to 90% by weight and more particularly in quantities of 60 to 80% by weight, expressed as mono- and diesters and based on mixture. The mixtures may also contain triesters and free citric acid as the balance to 100% by weight. However, the mixtures preferably contain little free citric acid, preferably less than 10% by weight, based on mixtures.

Accordingly, the preferred citric acid esters according to the invention are mainly partial esters of citric acid which still contain at least one free carboxyl group. The esters may therefore also be acidic esters or neutralization products thereof and X in formula (II) may be hydrogen or a cation. The partial esters are then preferably present in the form of alkali metal, alkaline earth metal, ammonium, alkylammonium, alkanolammonium and/or glucammonium salts (i.e. X=alkali metal, alkaline earth metal, ammonium, alkylammonium, alkanolammonium and/or glucammonium ion).

The α-hydroxycarboxylic acid esters of ethoxylated alcohols of component (b) according to the invention are used in the cosmetic preparations in quantities of 0.05 to 20% by weight, preferably in quantities of 0.5 to 10% by weight and, in one particularly preferred embodiment, in quantities of 0.5 to 6% by weight.

If the α-hydroxycarboxylic acid esters of ethoxylated alcohols of component (b) are used in combination with alkylether sulfates as component (a), it has been found that the conditioning effect of this composition is particularly pronounced where the mixing ratio of (a) to (b) is in the range from 1:10 to 10:1 and more particularly in the range from 1:1 to 10:1.

It may be assumed that the conditioning properties of the cosmetic preparations according to the present invention are attributable in particular to the presence of component (b). The α-hydroxycarboxylic acid esters of ethoxylated alcohols corresponding to formula (I):
R¹O(CH₂CH₂O)ₙH (I)
in which R¹ is a linear or branched alkyl and/or alkenyl group containing 6 to 22 carbon atoms and n is a number of 1 to 50,
may therefore be used as conditioning components in cosmetic preparations. Conditioning in the present context is understood to mean influencing of the feel, combability, softness, luster and smoothness of the skin and hair. Accordingly, the present invention also relates to the use of α-hydroxycarboxylic acid esters of ethoxylated alcohols corresponding to formula (I):
   R¹O(CH₂CH₂O)ₙH (I)
   in which R¹ is a linear or branched alkyl and/or alkenyl group containing 6 to 22 carbon atoms and n is a number of 1 to 50,
in cosmetic preparations for improving the feel of the skin and hair, for improving the wet and dry combability and antistatic behaviour of hair, for improving the softness of the skin and hair, for increasing the luster and/or smoothness of the skin and hair and as a moisturizing component for the hair. These effects are produced by the α-hydroxycarboxylic acid esters of ethoxylated alcohols according to the invention in particular because they prevent or at least reduce the absorption of alkyl ether sulfates onto the skin. Accordingly, the present invention also relates to the use of α-hydroxycarboxylic acid esters of ethoxylated alcohols corresponding to formula (I):
   R¹O(CH₂CH₂O)ₙH (I)
   in which R¹ is a linear or branched alkyl and/or alkenyl group containing 6 to 22 carbon atoms and n is a number of 1 to 50,
for preventing or reducing the absorption of alkyl ether sulfates onto human skin.
   Alkaline Earth Metal Ions

The preparations according to the invention additionally contain 0.005 to 1.5% by weight, preferably 0.005 to 1.0% by weight, more preferably 0.01 to 1.0% by weight and, in a most particularly preferred embodiment, 0.01 to 0.5% by weight alkaline earth metal ions. Calcium ions and/or magnesium ions are preferably used. It is important in this regard to bear in mind the fact that the water used in the production of the cosmetic preparation may already contain the required concentration of ions. By using these ions in the cosmetic preparations, the conditioning effect of the α-hydroxycarboxylic acid esters of ethoxylated alcohols corresponding to formula (I) is enhanced.

Silicone Compounds

The preparations according to the invention additionally contain 0.1 to 3% by weight of a silicone compound. Silicone compounds in the context of the present invention are, in particular, soluble or insoluble, volatile or nonvolatile polyorganosiloxanes, including polyalkyl siloxanes, polyaryl siloxanes, polyalkylaryl siloxanes, silicone rubbers and resins and chemically modified polyorganosiloxanes. A particularly preferred polyalkyl siloxane is CH₃-terminated polydimethyl siloxane. The chemically modified polyorganosiloxanes may be hydroxy-, thiol-, acyloxyacyl-, hydroxyacylamino-, amino- or alkenyloxy-modified. The amino- and hydroxy-functionalized polyorganosiloxanes are particularly preferred for the purposes of the invention.

In a particularly preferred embodiment, the cosmetic preparations according to the invention contain
(a) 9 to 20% by weight of at least one anionic, nonionic and/or amphoteric surfactant,
(b) 0.5 to 6% by weight of an α-hydroxycarboxylic acid ester of ethoxylated alcohols corresponding to formula (I):
   R¹O(CH₂CH₂O)ₙH (I)
   in which R¹ is a linear or branched alkyl and/or alkenyl group containing 6 to 22 carbon atoms and n is a number of 1 to 50,
(c) 0.01 to 0.5% by weight of at least one alkaline earth metal ion and
(d) 0.5 to 3% by weight of a silicone compound.
   Auxiliaries and Additives

The preparations according to the invention may additionally contain oil components, emulsifiers, pearlizing waxes, consistency factors, thickeners, superfatting agents, stabilizers, polymers, silicone compounds, fats, waxes, lecithins, phospholipids, biogenic agents and other active components, UV protection factors, antioxidants, antidandruff agents, film formers, swelling agents, hydrotropes, solubilizers, preservatives, perfume oils, dyes, etc. as further auxiliaries and additives.

Oil Components

Suitable oil components are, for example, Guerbet alcohols based on fatty alcohols containing 0.6 to 18 and preferably 8 to 10 carbon atoms, esters of linear C₆-22 fatty acids with linear or branched C₆-22 fatty alcohols or esters of branched C₆-13 carboxylic acids with linear or branched C₆-22 fatty alcohols such as, for example, myristyl myristate, myristyl palmitate, myristyl stearate, myristyl isostearate, myristyl oleate, myristyl behenate, myristyl erucate, cetyl myristate, cetyl palmitate, cetyl stearate, cetyl isostearate, cetyl oleate, cetyl behenate, cetyl erucate, stearyl myristate, stearyl palmitate, stearyl stearate, stearyl isostearate, stearyl oleate, stearyl behenate, stearyl erucate, isostearyl myristate, isostearyl palmitate, isostearyl stearate, isostearyl isostearate, isostearyl oleate, isostearyl behenate, isostearyl oleate, oleyl myristate, oleyl palmitate, oleyl stearate, oleyl isostearate, oleyl oleate, oleyl behenate, oleyl erucate, behenyl myristate, behenyl palmitate, behenyl stearate, behenyl isostearate, behenyl oleate, behenyl behenate, behenyl erucate, erucyl myristate, erucyl palmitate, erucyl stearate, erucyl isostearate, erucyl oleate, erucyl behenate and erucyl erucate. Also suitable are esters of linear C₆-22 fatty acids with branched alcohols, more particularly 2-ethyl hexanol, esters of C₁₈-38 alkylhydroxycarboxylic acids with linear or branched C₆-22 fatty alcohols, more especially Dioctyl Malate, esters of linear and/or branched fatty acids with polyhydric alcohols (for example propylene glycol, dimer diol or trimer triol) and/or Guerbet alcohols, triglycerides based on C₆-10 fatty acids, liquid mono-, di- and triglyceride mixtures based on C₆-18 fatty acids, esters of C₆-22 fatty alcohols and/or Guerbet alcohols with aromatic carboxylic acids, more particularly benzoic acid, esters of C₂-12 dicarboxylic acids with linear or branched alcohols containing 1 to 22 carbon atoms or polyols containing 2 to 10 carbon atoms and 2 to 6 hydroxyl groups, vegetable oils, branched primary alcohols, substituted cyclohexanes, linear and branched C₆-22 fatty alcohol carbonates, such as Dicaprylyl Carbonate (Cetiol® CC) for example, Guerbet carbonates based on C₆-18 and preferably C₈-10 fatty alcohols, esters of benzoic acid with linear and/or branched C₆-22 alcohols (for example Finsolv® TN), linear or branched, symmetrical or nonsymmetrical dialkyl ethers containing 6 to 22 carbon atoms per alkyl group, such as Dicaprylyl Ether (Cetiol® OE) for example, ring opening products of epoxidized fatty acid esters with polyols, silicone oils (cyclomethicone, silicon methicone types, etc.) and/or aliphatic or naphthenic hydrocarbons such as, for example, squalane, squalene or dialkyl cyclohexanes.

Fats and Waxes

Typical examples of fats are glycerides, i.e. solid or liquid, vegetable or animal products which consist essentially of mixed glycerol esters of higher fatty acids. Suitable waxes are inter alia natural waxes such as, for example, candelilla wax, carnauba wax, Japan wax, espartograss wax, cork wax, guaruma wax, rice oil wax, sugar cane wax, ouricury wax, montan wax, beeswax, shellac wax, spermaceti, lanolin (wool wax), uropygial fat, ceresine, ozocerite (earth wax), petrolatum, paraffin waxes and microwaxes; chemically modified waxes (hard waxes) such as, for example, montan ester waxes, sasol waxes, hydrogenated jojoba waxes and synthetic waxes such as, for example, polyalkylene waxes and polyethylene glycol waxes. Besides the fats, other suitable additives are fat-like substances, such as lecithins and phospholipids. Lecithins are known among experts as glycerophospholipids which are formed from fatty acids, glycerol, phosphoric acid and choline by esterification. Accordingly, lecithins are also frequently referred to by experts as phosphatidyl cholines (PCs). Examples of natural lecithins are the kephalins which are also known as phosphatidic acids and which are derivatives of 1,2-diacyl-sn-glycerol-3-phosphoric acids. By contrast, phospholipids are generally understood to be mono- and preferably diesters of phosphoric acid with glycerol (glycerophosphates) which are normally classed as fats. Sphingosines and sphingolipids are also suitable.

Pearlizing Waxes

Suitable pearlizing waxes are, for example, alkylene glycol esters, especially ethylene glycol distearate; fatty acid alkanolamides, especially cocofatty acid diethanolamide; partial glycerides, especially stearic acid monoglyceride; esters of polybasic, optionally hydroxysubstituted carboxylic acids with fatty alcohols containing 6 to 22 carbon atoms, especially long-chain esters of tartaric acid; fatty compounds, such as for example fatty alcohols, fatty ketones, fatty aldehydes, fatty ethers and fatty carbonates which contain in all at least 24 carbon atoms, especially laurone and distearylether; fatty acids, such as stearic acid, hydroxystearic acid or behenic acid, ring opening products of olefin epoxides containing 12 to 22 carbon atoms with fatty alcohols containing 12 to 22 carbon atoms and/or polyols containing 2 to 15 carbon atoms and 2 to 10 hydroxyl groups and mixtures thereof.

Consistency Factors and Thickeners

The consistency factors mainly used are fatty alcohols or hydroxyfatty alcohols containing 12 to 22 and preferably 16 to 18 carbon atoms and also partial glycerides, fatty acids or hydroxyfatty acids. A combination of these substances with alkyl oligoglucosides and/or fatty acid N-methyl glucamides of the same chain length and/or polyglycerol poly-12-hydroxystearates is preferably used. Suitable thickeners are, for example, Aerosil® types (hydrophilic silicas), polysaccharides, more especially xanthan gum, guar-guar, agar-agar, alginates and tyloses, carboxymethyl cellulose and hydroxyethyl and hydroxypropyl cellulose, also relatively high molecular weight polyethylene glycol monoesters and diesters of fatty acids, polyacrylates (for example Carbopols® and Pemulen types [Noveon]; Synthalens® [Sigma]; Keltrol types [Kelco]; Sepigel types [Seppic]; Salcare types [Allied Colloids]), polyacrylamides, polymers, polyvinyl alcohol and polyvinyl pyrrolidone. Other consistency factors which have proved to be particularly effective are bentonites, for example Bentone® Gel VS-5PC (Rheox) which is a mixture of cyclopentasiloxane, Disteardimonium Hectorite and propylene carbonate. Other suitable consistency factors are surfactants such as, for example, ethoxylated fatty acid glycerides, esters of fatty acids with polyols, for example pentaerythritol or trimethylol propane, narrow-range fatty alcohol ethoxylates or alkyl oligoglucosides and electrolytes, such as sodium chloride and ammonium chloride. Sodium polynaphthalene sulfates, acrylate/aminoacrylate/C₁₀₋₃₀ alkyl PEG 20 itaconate copolymers and polyacrylamidomethyl propanesulfonic acid are also mentioned.

Superfatting Agents

Superfatting agents may be selected from such substances as, for example, lanolin and lecithin and also polyethoxylated or acylated lanolin and lecithin derivatives, polyol fatty acid esters, monoglycerides and fatty acid alkanolamides, the fatty acid alkanolamides also serving as foam stabilizers.

Stabilizers

Metal salts of fatty acids such as, for example, magnesium, aluminum and/or zinc stearate or ricinoleate may be used as stabilizers.

Film Formers

Standard film formers are, for example, chitosan, microcrystalline chitosan, quaternized chitosan, polyvinyl pyrrolidone, vinyl pyrrolidone/vinyl acetate copolymers, polymers of the acrylic acid series, quaternary cellulose derivatives, collagen, hyaluronic acid and salts thereof and similar compounds.

Antidandruff Agents

Suitable antidandruff agents are Pirocton Olamin (1-hydroxy-4-methyl-6-(2,4,4-trimethylpentyl)-2-(1H)-pyridinone monoethanolamine salt), Crinipan® AD (climbazole), Ketoconazol® (4-acetyl-1-{4-[2-(2,4-dichlorophenyl) r-2-(1H-imidazol-1-ylmethyl)-1,3-dioxylan-c-4-ylmethoxy-phenyl}-piperazine, ketoconazole, elubiol, selenium disulfide, colloidal sulfur, sulfur polyethylene glycol sorbitan monooleate, sulfur ricinol polyethoxylate, sulfur tar distillate, salicylic acid (or in combination with hexachlorophene), undecylenic acid, monoethanolamide sulfosuccinate Na salt, Lamepon® UD (protein/undecylenic acid condensate), zinc pyrithione, aluminum pyrithione and magnesium pyrithione/dipyrithione magnesium sulfate.

Swelling Agents

Suitable swelling agents for aqueous phases are montmorillonites, clay minerals, Pemulen and alkyl-modified Carbopol types (Noveon).

Hydrotropes

In addition, hydrotropes, for example ethanol, isopropyl alcohol or polyols, may be used to improve flow behavior. Suitable polyols preferably contain 2 to 15 carbon atoms and at least two hydroxyl groups. The polyols may contain other functional groups, more especially amino groups, or may be modified with nitrogen. Typical examples are
glycerol;
alkylene glycols such as, for example, ethylene glycol, diethylene glycol, propylene glycol, butylene glycol, hexylene glycol and polyethylene glycols with an average molecular weight of 100 to 1000 dalton;
technical oligoglycerol mixtures with a degree of self-condensation of 1.5 to 10 such as, for example, technical diglycerol mixtures with a diglycerol content of 40 to 50% by weight;
methylol compounds such as, in particular, trimethylol ethane, trimethylol propane, trimethylol butane, pentaerythritol and dipentaerythritol;
lower alkyl glucosides, particularly those containing 1 to 8 carbon atoms in the alkyl group, for example methyl and butyl glucoside;
sugar alcohols containing 5 to 12 carbon atoms, for example sorbitol or mannitol,
sugars containing 5 to 12 carbon atoms, for example glucose or sucrose;
amino sugars, for example glucamine;
dialcoholamines, such as diethanolamine or 2-aminopropane-1,3-diol.
   Active Components

The active components used may be proteins and protein derivatives such as, for example, protein condensates or protein hydrolyzates.

Preservatives

Suitable preservatives are, for example, phenoxyethanol, formaldehyde solution, parabens, pentanediol or sorbic acid and the silver complexes known under the name of Surfacine®.

Perfume Oils and Aromas

Suitable perfume oils are mixtures of natural and synthetic perfumes. Natural perfumes include the extracts of blossoms (lily, lavender, rose, jasmine, neroli, ylang-ylang), stems and leaves (geranium, patchouli, petitgrain), fruits (anise, coriander, caraway, juniper), fruit peel (bergamot, lemon, orange), roots (nutmeg, angelica, celery, cardamom, costus, iris, calmus), woods (pinewood, sandalwood, guaiac wood, cedarwood, rosewood), herbs and grasses (tarragon, lemon grass, sage, thyme), needles and branches (spruce, fir, pine, dwarf pine), resins and balsams (galbanum, elemi, benzoin, myrrh, olibanum, opoponax). Animal raw materials, for example civet and beaver, may also be used. Typical synthetic perfume compounds are products of the ester, ether, aldehyde, ketone, alcohol and hydrocarbon type. Examples of perfume compounds of the ester type are benzyl acetate, phenoxyethyl isobutyrate, p-tert-butyl cyclohexylacetate, linalyl acetate, dimethyl benzyl carbinyl acetate, phenyl ethyl acetate, linalyl benzoate, benzyl formate, ethylmethyl phenyl glycinate, allyl cyclohexyl propionate, styrallyl propionate and benzyl salicylate. Ethers include, for example, benzyl ethyl ether while aldehydes include, for example, the linear alkanals containing 8 to 18 carbon atoms, citral, citronellal, citronellyl-oxyacetaldehyde, cyclamen aldehyde, hydroxycitronellal, lilial and bourgeonal. Examples of suitable ketones are the ionones, α-isomethylionone and methyl cedryl ketone. Suitable alcohols are anethol, citronellol, eugenol, isoeugenol, geraniol, linalool, phenylethyl alcohol and terpineol. The hydrocarbons mainly include the terpenes and balsams. However, it is preferred to use mixtures of different perfume compounds which, together, produce an agreeable perfume. Other suitable perfume oils are essential oils of relatively low volatility which are mostly used as aroma components. Examples are sage oil, camomile oil, clove oil, melissa oil, mint oil, cinnamon leaf oil, lime-blossom oil, juniper berry oil, vetiver oil, olibanum oil, galbanum oil, ladanum oil and lavendin oil. The following are preferably used either individually or in the form of mixtures: bergamot oil, dihydromyrcenol, lilial, lyral, citronellol, phenylethyl alcohol, α-hexylcinnamaldehyde, geraniol, benzyl acetone, cyclamen aldehyde, linalool, Boisambrene Forte, Ambroxan, indole, hedione, sandelice, citrus oil, mandarin oil, orange oil, allylamyl glycolate, cyclovertal, lavendin oil, clary oil, β-damascone, geranium oil bourbon, cyclohexyl salicylate, Vertofix Coeur, Iso-E-Super, Fixolide NP, evernyl, iraldein gamma, phenylacetic acid, geranyl acetate, benzyl acetate, rose oxide, romillat, irotyl and floramat.

Suitable aromas are, for example, peppermint oil, spearmint oil, aniseed oil, Japanese anise oil, caraway oil, eucalyptus oil, fennel oil, citrus oil, wintergreen oil, clove oil, menthol and the like.

Dyes

Suitable dyes are any of the substances suitable and approved for cosmetic purposes. Examples include cochineal red A (C.I. 16255), patent blue V (C.I. 42051), indigotin (C.I. 73015), chlorophyllin (C.I. 75810), quinoline yellow (C.I. 47005), titanium dioxide (C.I. 77891), indanthrene blue RS(C.I. 69800) and madder lake (C.I. 58000). Luminol may also be present as a luminescent dye. These dyes are normally used in concentrations of 0.001 to 0.1% by weight, based on the mixture as a whole.

| Formulation No.: 04/136 | 1 | 2 | 3 | 4 | 5 | 6 | 7 | 8 |
|---|---|---|---|---|---|---|---|---|
| Texapon ® N 70 | 19.5 | 21.0 | 17.5 | 15.6 | 19.4 | 18.6 | 18.2 | 19.7 |
| Sodium Laureth Sulfate 70% as | | | | | | | | |
| Dehyton ® PK 45 | — | — | 5.0 | — | — | 2.5 | — | 1.5 |
| Cocamidopropyl Betaine 40% as | | | | | | | | |
| Dehyton ® DC | — | — | — | 6.0 | — | — | 3.5 | — |
| Disodium Cocoamphodiacetate 40% as | | | | | | | | |
| Plantapon ® ACG 35 | — | — | — | — | 6.5 | — | — | 2.0 |
| Disodium Cocoyl Glutamate 35% as | | | | | | | | |
| Plantapon ® LC 7 | 3.1 | 2.4 | 3.3 | 2.3 | 2.1 | 3.4 | 2.2 | 2.6 |
| Laureth-7 Citrate 100% as | | | | | | | | |
| Polymer JR 400 | — | — | 0.03 | 0.07 | 0.05 | — | — | — |
| Polyquaternium-10 100% as | | | | | | | | |
| Cosmedia ® Guar C 261N | — | — | — | — | — | — | — | 0.05 |
| Guar Hydroxypropyltrimonium Chloride | | | | | | | | |
| 100% as | | | | | | | | |
| Gluadin ® WQ | — | — | — | 0.5 | — | — | — | — |
| Laurdimonium Hydroxypropyl Hydrolyzed | | | | | | | | |
| Wheat Protein 33% as | | | | | | | | |
| Dehyquart ® E CA | — | — | — | — | 1.0 | — | — | — |
| Hydroxycetyl Hydroxyethyl Dimonium | | | | | | | | |
| Chloride 23% as | | | | | | | | |
| Cetiol ® LDO | — | — | — | — | — | 1.0 | 1.0 | 0.7 |
| Dicaprylyl Ether and Lauryl Alcohol | | | | | | | | |
| 100% as | | | | | | | | |
| Lamesoft ® TM Benz | — | — | — | — | — | — | — | 3.5 |
| Glycol Distearate and Coco Glucoside | | | | | | | | |
| and Glyceryl Oleate and Glycery | | | | | | | | |
| Stearate | | | | | | | | |
| DC 193 Surfactant | 2.5 | 2.0 | 1.5 | 1.0 | 1.0 | 0.5 | 0.5 | — |
| PEG-12 Dimethicone | | | | | | | | |
| DC 1784 Emulsion | — | — | — | — | — | — | — | 1.0 |
| Dimethiconol (and) TEA- | | | | | | | | |
| Dodecylbenzenesulfonate 50% as | | | | | | | | |
| Cosmedia ® HSP-1180 | 1.0 | 1.5 | — | — | — | — | — | — |
| Polyacrylaminomethylpropane Sulfonic | | | | | | | | |
| Acid | | | | | | | | |
| Carbopol Aqua SF-1 | — | — | — | — | — | — | — | 3.0 |
| Acrylates Copolymer 30% as | | | | | | | | |
| Structure Plus | — | — | 2.5 | 3.0 | 3.5 | 3.0 | 3.0 | — |
| Acrylates/Aminoacrylates/C10-30 Alkyl | | | | | | | | |
| PEG-20 Itaconate Copolymer 20% as | | | | | | | | |
| Perfume | 0.3 | 0.3 | 0.3 | 0.3 | 0.3 | 0.3 | 0.3 | 0.3 |
| Preservative | 0.7 | 0.7 | 0.7 | 0.7 | 0.7 | 0.7 | 0.7 | 0.7 |
| CaCl₂.2H₂O | 0.16 | — | 0.22 | 0.2 | — | 0.19 | — | 0.1 |
| MgCl₂ | 0.1 | 0.22 | — | — | 0.19 | — | 0.25 | 0.11 |
| NaCl | 0.65 | 0.5 | 0.35 | 0.45 | 0.55 | 0.5 | 0.5 | — |
| NaOH 40% | 0.15 | 0.11 | 0.17 | 0.1 | 0.08 | 0.18 | 0.09 | 0.28 |
| Water | to 100 | | | | | | | |
| pH | 5.2 | 5.3 | 5.7 | 5.5 | 5.3 | 5.4 | 5.4 | 6.6 |
| Viscosity in mPa · s | 4450 | 5200 | 4850 | 6600 | 7550 | 6750 | 5900 | 8100 |

| Formulation no.: 04/136 | 9 | 10 | 11 | 12 | 13 | 14 | 15 | 16 | 17 |
|---|---|---|---|---|---|---|---|---|---|
| Texapon ® N 70 | 18.6 | 11.5 | 8.4 | 15.3 | 14.8 | 20.1 | 18.8 | 21.2 | 19.8 |
| Sodium Laureth Sulfate 70% as | | | | | | | | | |
| Dehyton ® PK 45 | — | 7.8 | 9.3 | 3.8 | 4.5 | — | — | — | — |
| Cocamidopropyl Betaine 40% as | | | | | | | | | |
| Dehyton ® DC | 4.0 | — | 5.4 | 2.2 | — | — | — | — | — |
| Disodium Cocoamphodiacetate 40% as | | | | | | | | | |
| Plantapon ® ACG 35 | 3.0 | 5.6 | 6.7 | — | 3.6 | — | 2.5 | 2.0 | 3.5 |
| Disodium Cocoyl Glutamate 35% as | | | | | | | | | |
| Plantapon ® LC 7 | 3.1 | 4.2 | 2.8 | 4.6 | 5.8 | 3.5 | 2.8 | 2.5 | 3.3 |
| Laureth-7 Citrate 100% as | | | | | | | | | |
| Polymer JR 400 | — | — | — | — | — | — | — | — | — |
| Polyquaternium-10 100% as | | | | | | | | | |
| Cosmedia ® Guar C 261N | 0.03 | — | — | — | — | — | — | — | — |
| Guar Hydroxypropyltrimonium Chloride | | | | | | | | | |
| 100% as | | | | | | | | | |
| Gluadin ® WQ | 0.5 | — | 2.0 | — | — | — | — | — | — |
| Laurdimonium Hydroxypropyl | | | | | | | | | |
| Hydrolyzed Wheat Protein 33% as | | | | | | | | | |
| Dehyquart ® E CA | — | 0.8 | — | 0.5 | — | — | — | — | — |
| Hydroxycetyl Hydroxyethyl Dimonium | | | | | | | | | |
| Chloride 23% as | | | | | | | | | |
| Cetiol ® LDO | 0.5 | — | — | — | 0.55 | — | — | — | — |
| Dicaprylyl Ether and Lauryl Alcohol | | | | | | | | | |
| 100% as | | | | | | | | | |
| Lamesoft ® TM Benz | 4.0 | 4.6 | 3.8 | 3.2 | 3.8 | 4.5 | 5.0 | 4.2 | 4.4 |
| Glycol Distearate and Coco Glucoside | | | | | | | | | |
| and Glyceryl Oleate and Glycery | | | | | | | | | |
| Stearate | | | | | | | | | |
| DC 193 Surfactant | — | — | — | — | — | — | 2.8 | — | — |
| PEG-12 Dimethicone | | | | | | | | | |
| DC 1784 Emulsion | 1.5 | 2.5 | 3.0 | 1.8 | 2.0 | 3.3 | | 2.2 | 1.8 |
| Dimethiconol (and) TEA- | | | | | | | | | |
| Dodecylbenzenesulfonate 50% as | | | | | | | | | |
| Cosmedia HSP-1180 | — | — | — | — | — | — | 2.1 | — | — |
| Polyacrylaminomethylpropane Sulfonic | | | | | | | | | |
| Acid | | | | | | | | | |
| Carbopol Aqua SF-1 | 3.0 | — | — | — | — | 2.5 | — | — | — |
| Acrylates Copolymer 30% as | | | | | | | | | |
| Structure Plus | — | 3.4 | 3.3 | 3.0 | 3.5 | — | — | 4.5 | 3.8 |
| Acrylates/Aminoacrylates/C10-30 Alkyl | | | | | | | | | |
| PEG-20 Itaconate Copolymer 20% as | | | | | | | | | |
| Perfume | 0.3 | 0.3 | 0.3 | 0.3 | 0.3 | 0.3 | 0.3 | 0.3 | 0.3 |
| Preservative | 0.7 | 0.7 | 0.7 | 0.7 | 0.7 | 0.7 | 0.7 | 0.7 | 0.7 |
| CaCl₂.2H₂O | 0.05 | 0.18 | — | 0.19 | 0.31 | 0.38 | — | — | — |
| MgCl₂ | 0.18 | 0.05 | 0.32 | 0.03 | — | — | 0.49 | 0.55 | 1.1 |
| NaCl | — | — | — | — | — | — | — | — | — |
| NaOH 40% | 0.31 | 0.21 | 0.13 | 0.26 | 0.38 | 0.33 | 0.24 | 0.22 | 0.24 |
| Water | to 100 | | | | | | | | |
| pH | 6.5 | 5.6 | 5.3 | 5.6 | 5.5 | 6.5 | 5.5 | 5.3 | 5.6 |
| Viscosity in mPa · s | 8800 | 9440 | 8660 | 6930 | 7540 | 7340 | 7650 | 8750 | 12500 |

Sensory Assessment

The conditioning properties of two surfactant mixtures were compared with one another by sensory assessment. After the sample composition had been completely absorbed onto the skin, the upper side of the middle and index fingers was slowly drawn without pressing over the treated inside of the forearm. If the skin surface was felt to be without friction, this corresponded to a soft skin feel.

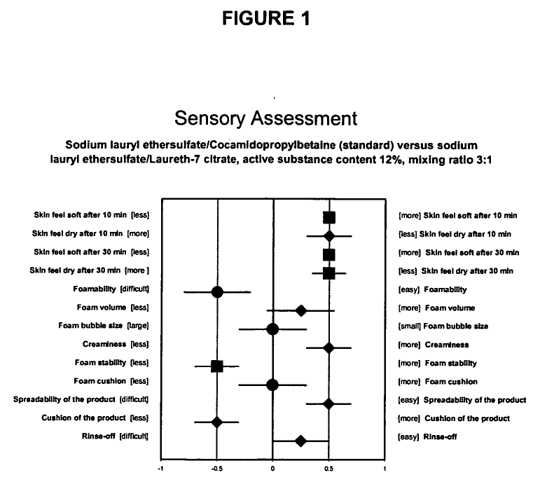

It can be seen from FIG. 1 that skin treated with the co-surfactant of the invention, Laureth-7 Citrate, is felt to be softer than skin treated with a standard co-surfactant, Cocamidopropylbetaine. In FIG. 1, marks in the left column favor the standard co-surfactant and marks in the right column favor the inventive co-surfactant for that property. In FIG. 1, square marks indicate a high degree of significance; diamonds indicate a medium degree of significance and circles indicate a low degree of significance (as indicated also by the length of the horizontal lines through the marks).

Half Head Test

Each test was conducted on 10 volunteers by trained and skilled people. Each half of the head was prewashed and then treated with the corresponding products. During the test, performance properties, such as skin feel, were evaluated first, followed by such parameters as combability, feel, luster and volume.

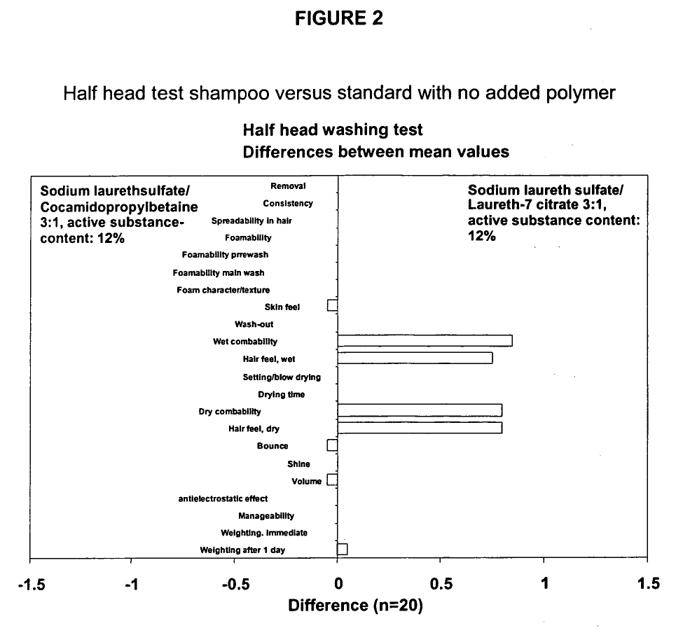

It can be seen from FIG. 2 that the preparation containing the α-hydroxycarboxylic acid ester co-surfactant of the invention (Laureth-7-citrate) has clearly superior conditioning properties to the preparation containing only a standard co-surfactant (Cocamidopropylbetaine) and no added polymer. The hair can be combed far better both in the dry and in the wet state; the feel properties are also found to be distinctly better than those of hair treated with the standard preparation. In FIG. 2, a bar on one side or the other shows a result favoring the composition on that side for that property.

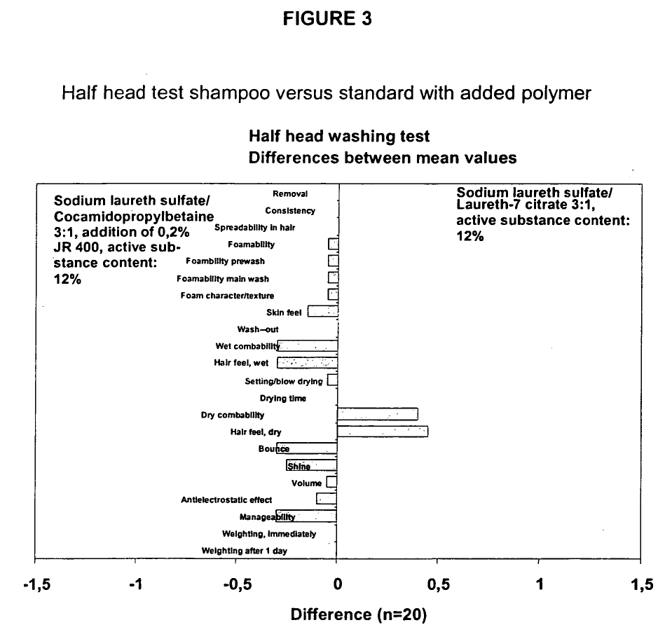

FIG. 3 clearly shows that the preparation containing the α-hydroxycarboxylic acid ester co-surfactant of the invention (Laureth-7-citrate) leads to improved combability of dry hair and to a more pleasant feel of the hair over the standard co-surfactant (Cocamidopropylbetaine) and a polymer. In FIG. 3, a bar on one side or the other shows a result favoring the composition on that side for that property.

Absorption Behavior

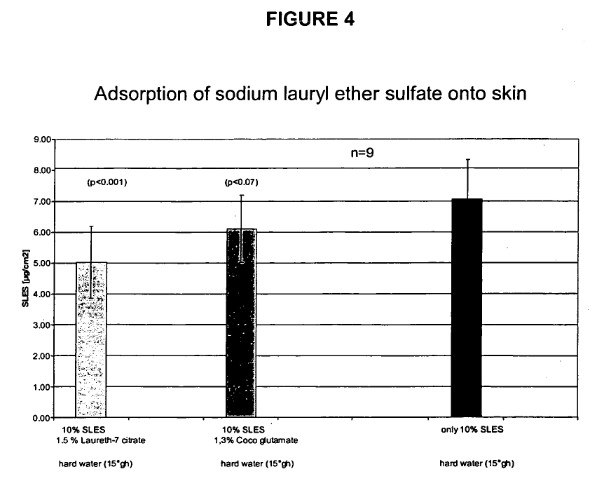

FIG. 4 shows that, where α-hydroxycarboxylic acid esters of ethoxylated alcohols of formula (I) (Laureth-7 Citrate) according to the invention are present, the adsorption of sodium lauryl ether sulfate is prevented considerably more effectively than with a standard co-surfactant (cocoglutamate).

What is claimed is:

1. A cosmetic composition comprising
(a) 0.1 to 30% by weight of at least one surfactant selected from the group consisting of anionic, nonionic and amphoteric surfactants,
(b) 0.05 to 20% by weight of a partial or full α-hydroxycarboxylic acid ester of ethoxylated alcohols of formula (I):
R¹O(CH₂CH₂O)ₙH (I)
in which R¹ is a linear or branched alkyl or alkenyl group containing 6 to 22 carbon atoms and n is a number of 1 to 50,
(c) 0.005 to 1.5% by weight of at least one alkaline earth metal ion, and
(d) 0.1 to 3% by weight of a silicone compound.

2. A cosmetic composition according to claim 1, wherein the at least one surfactant of component (a) is selected from the group consisting of alkyl ether sulfates, alkyl amidobetaines, acylated amino acids, alk(en)yl oligoglycosides, alkyl glucose carboxylates and alkyl amphoacetates.

3. A cosmetic composition according to claim 1, wherein the ester of component (b) is a partial ester containing at least one free carboxylic acid.

4. A cosmetic composition according to claim 3, wherein the at least one free carboxylic acid is present in the form of the alkali metal, alkaline earth metal, ammonium, alkylammonium, or glucammonium salt or mixtures of such salts.

5. A cosmetic composition according to claim 1, wherein the α-hydroxycarboxylic acid ester of ethoxylated alcohols of formula (I) is selected from esters of citric acid.

6. A cosmetic composition according to claim 1, wherein component (b) is selected from citric acid esters which are mixtures of isomeric compounds corresponding to general formula (II): 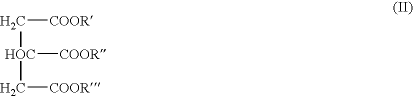
in which R′, R″, R′″ independently stand for X or an ethoxylated alkyl group R¹, the distribution of the substituents R′, R″ and R′″ having to be such that the ratio by weight of monoester to diester is in the range from 3:1 to 10:1, wherein X represents an alkali metal, alkaline earth metal, ammonium, alkylammonium, alkanolammonium or glucammonium ion or mixtures of such ions and R¹ has the meaning defined for formula (I).

7. A cosmetic composition according to claim 6, wherein the citric acid ester contains at least one free carboxylic acid.

8. A cosmetic composition according to claim 7, wherein the at least one free carboxylic acid is present in the form of the alkali metal, alkaline earth metal, ammonium, alkylammonium, or glucammonium salt or mixtures of such salts.

9. A cosmetic composition according to claim 1, wherein component (b) is present in quantities of 0.5 to 10% by weight.

10. A cosmetic composition according to claim 1, wherein the at least one alkaline earth metal ion of component (c) is selected from calcium, magnesium and mixtures thereof.

11. A cosmetic composition according to claim 1, wherein the silicone compound of component (d) is an amino- and hydroxy-functionalized polyorganosiloxane.

12. A process for improving the conditioning properties of a cosmetic composition for the skin or hair, said process comprising combining a partial or full α-hydroxycarboxylic acid ester of ethoxylated alcohols of formula (I):
R¹O(CH₂CH₂O)ₙH (I)
in which R¹ is a linear or branched alkyl or alkenyl group containing 6 to 22 carbon atoms and n is a number of 1 to 50, with such cosmetic composition.

13. A process according to claim 12, wherein the α-hydroxycarboxylic acid ester of ethoxylated alcohols of formula (I) comprises 0.05 to 20% by weight of the cosmetic composition.

14. A process according to claim 12, wherein the α-hydroxycarboxylic acid ester of ethoxylated alcohols of formula (I) is a partial ester containing at least one free carboxylic acid.

15. A process according to claim 13, wherein the at least one free carboxylic acid is present in the form of the alkali metal, alkaline earth metal, ammonium, alkylammonium, or glucammonium salt or mixtures of such salts.

16. A process according to claim 12, wherein the α-hydroxycarboxylic acid ester of ethoxylated alcohols of formula (I) is selected from such esters of citric acid.

17. A process according to claim 12, wherein the α-hydroxycarboxylic acid ester of ethoxylated alcohols of formula (I) is selected from citric acid esters which are mixtures of isomeric compounds corresponding to general formula (II): 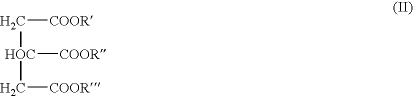
in which R′, R″, R′″ independently stand for X or an ethoxylated alkyl group R¹, the distribution of the substituents R′, R″ and R′″ having to be such that the ratio by weight of monoester to diester is in the range from 3:1 to 10:1, wherein X represents an alkali metal, alkaline earth metal, ammonium, alkylammonium, alkanolammonium or glucammonium ion or mixtures of such ions and R¹ has the meaning defined for formula (I).

18. A process according to claim 17, wherein the citric acid esters contain at least one free carboxylic acid.

19. A process according to claim 18, wherein the at least one free carboxylic acid is present in the form of the alkali metal, alkaline earth metal, ammonium, alkylammonium, or glucammonium salt or mixtures of such salts.

20. A process for preventing or reducing the absorption of alkyl ether sulfates onto the human skin, said process comprising applying to the skin a cosmetic composition containing a partial or full α-hydroxycarboxylic acid ester of ethoxylated alcohols of formula (I):
R¹O(CH₂CH₂O)ₙH (I)
in which R¹ is a linear or branched alkyl or alkenyl group containing 6 to 22 carbon atoms and n is a number of 1 to 50.

21. A process according to claim 20, wherein the α-hydroxycarboxylic acid ester of ethoxylated alcohols of formula (I) comprises 0.05 to 20% by weight of the cosmetic composition.

22. A process according to claim 20, wherein the α-hydroxycarboxylic acid ester of ethoxylated alcohols of formula (I) Is a partial ester containing at least one free carboxylic acid.

23. A process according to claim 22, wherein the at least one free carboxylic acid is present in the form of the alkali metal, alkaline earth metal, ammonium, alkylammonium, or glucammonium salt or mixtures of such salts.

24. A process according to claim 20, wherein the α-hydroxycarboxylic acid ester of ethoxylated alcohols of formula (I) is selected from esters of citric acid.
